(19) United States
(12) Patent Application Publication
(10) Pub. No.: US 2008/0220037 A1
(43) Pub. Date: Sep. 11, 2008

(54) Biphosphonic Compounds For Preventing Or Limiting Fixing Of Macromolecules, Microorganisms And Biofilm On Solid, In Particular Metal Or Mineral Surfaces

(75) Inventors: Benoit Denizot, Bonneville (FR); Francois Hindre, Angers (FR); David Portet, Briollay (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignees: Surfactis Technologies, Angers (FR); Institut National de la Sante et de la Recherche M, Paris (FR); Universite D'Angers, Angers (FR)

(21) Appl. No.: 11/791,291

(22) PCT Filed: Nov. 21, 2005
(86) PCT No.: PCT/EP2005/056120

§ 371 Date: Sep. 24, 2007

(30) Foreign Application Priority Data
Nov. 22, 2004 (FR) 0412358

(51) Int. Cl. A01N 25/34; C07F 9/38; A61K 31/664; A61K 8/24; A61K 9/68; A01N 57/18

(52) U.S. Cl. 424/411; 562/13; 514/77; 424/57; 424/48

(57) ABSTRACT

The invention concerns biphosphonic compounds of formula (I); microbiological anti-contamination compositions containing same, and their use for preventing or limiting fixing of macromolecules, micro-organisms and biofilm on solid surfaces, such as metal or mineral surfaces.

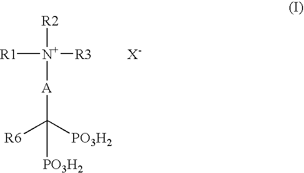

The invention relates to the field of the protection of solid surfaces, such as metal or mineral surfaces, against macromolecular and microbiological contamination, in particular bacterial contamination.

The term “microbiological contamination” is intended to mean contamination with microorganisms.

The invention relates in particular to novel bisphosphonic compounds, to the anti-contamination compounds containing them, and to their use for limiting the attachment of macromolecules and microorganisms, in particular bacteria, on surfaces, such as metal or mineral surfaces.

In the food or medical industry, surfaces represent a considerable source of bacteriological contamination.

For example, in industries which use fermentations or reactors as biotechnology, the adsorption of macro-molecules onto the walls of the containers can result in the denaturation of these compounds, which has a deleterious effect in the case of productions of high-value proteins (such as cytokines, for example) by means of fermenters.

In other situations, the polymeric layer formed can itself be potentially pathogenic, as in the case of adsorbed and partially denatured prions, the complete removal of which from the surface is difficult to obtain.

Microorganisms, and in particular bacteria, are capable of colonizing varied surfaces and of forming real assemblies. These surface-colonization phenomena are responsible for the formation of the biofilm which constitutes a considerable source of microbiological contamination.

The formation of biofilms on a surface involves various successive phenomena.

The precursor phenomenon is the attachment or the adsorption of macromolecules by virtue of their denaturation and their spreading out on contact with the surface. These polymeric macromolecules are of protein or polysaccharide, or even polyphenolic, type. In general, they are of biological origin, in particular of bacterial origin. By attaching to the surface, the macromolecules form a potentially pathogenic polymeric layer, as in the case of adsorbed prions.

Numerous microorganisms, such as protozoa, bacteria, fungi or algae, will then be able to develop in this polymeric layer due to the presence of hydrophobic zones, of amine or sulfide groups or of monosaccharide or polysaccharide sites. Once attached, under favorable conditions, these microorganisms will not only multiply, but will also secrete other polymers, thus constructing a film-type matrix, known as a biofilm. This film is known, for example, to be an essential factor in the contamination of air-conditioning plants by legionellae, allowing dissemination of these microorganisms in high concentration.

Under specific conditions, such as in marine or river environments, this microbiological film will serve as a support for multicellular plant or animal organisms, resulting in thick films. One example is that of the contamination of boat hulls, resulting in a considerable increase in their resistance to forward movement.

In general, these phenomena as a whole are often known as “fouling” or “biofouling”, which can be defined in the following way: all the successive processes that result in the colonization of surfaces by micro-organisms or even multicellular organisms.

A similar phenomenon is that of implanted prostheses, which become rapidly covered with the macromolecules present in the media in contact, thereby subsequently serving as a support for the invasion of migrating cells.

The structure of the biofilm is in general porous and allows water and nutrients to circulate, thereby allowing microorganism colony renewal and development. This macromolecular layer also makes it possible to protect the microorganisms against outside attacks (biocides, antibiotics, antiseptics) by slowing down the access of products which are biologically active with respect to the cells. During the macromolecular adsorption step, various forces of attraction are involved depending on the type of surface. Those which are generally involved in this process are of the electrostatic, ionic, Van Der Waals, hydrogen bond or hydrophobic interaction type. The adsorption thus depends on the forces of attraction and repulsion which exist between the macromolecules and the surface. In addition, many factors must be taken into account, such as the surface tension of the support (modulated by the presence or absence of surfactants), or else the uneven distribution of electric charges on the surface.

After this adsorption phase, the attachment per se of the microorganism depends on its type, on the size of its population in the medium, on the duration of its growth phase, and on the cellular deformation. The latter also depends on the dispersing medium: temperature of the solution, pH, electrolyte concentration and nutrient availability. Finally, the force of attachment also depends on the charge of the surface and on the duration of contact.

After attachment, the microcolonies form on the surface. The growth and then the confluence thereof rapidly results in the formation of a thin and superficial coating at the start, which thickens with the microbiological growth, to reach a few millimeters of thickness: this is the biofilm. The macromolecular adhesion is therefore an essential step in the biofilm formation process.

The most well-known biofilm is probably the dental biofilm, commonly called dental plaque, a complex ecosystem of the oral cavity in humans, which is responsible for dental caries, recurrences of caries, periodontal diseases and peri-implantitis, which threaten the longevity of the biomaterial.

Biocidal chemical agents, such as chlorine or antibiotics, often prove to be ineffective or insufficient against the formation of the biofilm on surfaces. In fact, the chlorination of a biofilm reaches only the outer part of the biofilm and leave intact a layer of bacteria capable of developing rapidly again. The use of chlorine to remove the biofilm is only effective if the latter is removed from the surface manually.

Antibiotics are not satisfactory either, since they are liable to bring about the dissemination of resistance genes in microorganisms, in particular bacteria, thereby gradually rendering them inactive. It is common to see biocidal products that are active in vitro against isolated bacteria, become completely inactive against sessile bacteria.

Moreover, the use of repellent molecules is found to be effective only for evolved multicellular organisms.

At the current time, research is therefore directed toward the development of products that are active against the biofilms, and no longer only against the development of the microorganisms or the bacteria themselves. Two possibilities exist for combating surface contamination:
A direct enzymatic action which allows degradation of the structure of the biofilm, thereby facilitating detachment thereof.
A limitation of the adhesion of the macromolecules to the surfaces in order to prevent the development of microorganism colonies and to facilitate the cleaning of these surfaces.

In the present invention, the approach selected consists in rendering the physicochemical properties of solid surfaces, in particular the forces of attraction of these surfaces, unfavorable to macromolecular adhesion or adsorption, and thus in limiting or even preventing the attachment of microorganisms, in particular of bacteria, to these surfaces.

The difficulty of this approach lies in the identification of a compound which is both capable of rapidly and effectively attaching to or adsorbing onto, in a long-lasting manner, the surface to be protected, and which prevents or limits the attachment of microorganisms to this surface, by virtue of the creation of an interface, between the surface and the outside medium, which is unfavorable to the adhesion and to the development of microorganisms.

A known approach for limiting the adsorption of macro-molecules is coating with hydrophilic polymers by adsorption and/or grafting. The brownien movement of the chains of said polymers repels the molecules liable to adsorb. In this range of molecules are natural polymers such as certain polysaccharides (dextran, for example) and proteins (the most common example of which is albumin), but also synthetic polymers such as poly-ethylene glycols (PEGs). Despite its undeniable advantages and its proven effectiveness, this polymeric brush effect has certain limitations: the surface density must be neither too low (in this case, there are holes in the layer and therefore possibilities of adsorption) nor too high (the chains no longer exhibit the sufficient freedom to have a brownien movement for repelling the other molecules). In addition, the interactions between the immobilized macromolecules and the molecules in solution, regardless of whether or not they are specific in nature, must be weak. This point in fact excludes all highly electrostatically charged polymers that can enter into strong interactions with macromolecules that have a high opposite charge. From a practical point of view, the preparation of a coating with suitable and long-lasting properties is in general difficult to carry out, in particular for widespread surfaces. Means for succeeding in obtaining layers with suitable properties are proposed in this patent.

A few years ago, it was shown that surfaces expressing phosphorylcholine groups in sufficient density could considerably limit protein adsorption (Biomaterials, 2002, 23, 3699-3710). This was in particular shown for phosphatidylcholine-based liposomes and in the protection of vascular prostheses coated with polymers bearing phosphorylcholine groups. Similarly, it is possible to considerably reduce the adsorption of proteins onto “gold”-type metal supports by using molecules, comprising phosphorylcholine groups, attached to the support via thiol groups. The mechanism implicated in these effects appears to be linked to the intrinsic strong hydrophilicity of the grafted phosphorylcholine and to the presence of an electric dipole, in the absence of Lewis acid-base groups capable of entering into hydrogen bonds. This hypothesis would explain why zwitterionic molecules such as sulfobetaines are also capable of having an anti-adsorption effect.

U.S. Pat. No. 5,888,405 proposes the use of aminophosphonates for modifying surface properties and thereby impairing the biofouling phenomenon. However, the forces of adsorption of phosphonates onto iron oxide-type metal supports are relatively discrete (J. Coll. Interface Sci., 238(1):37-42) and imply a limited action.

In the literature, no available molecule exists that limits protein adsorption and is capable of attaching strongly to supports such as steel, aluminum, calcium salts or modified glass.

The invention aims to overcome the drawbacks of the prior art by proposing compounds and compositions that are particularly effective and suitable for the reduction of biofouling (in the broad sense of the term), according to various possible applications, in particular pastes and gels, aqueous, alcoholic or organic solutions, suspensions, foams, powders, aerosols, etc.

The invention aims to define the manufacturing protocol most suitable for molecules that are effective in these applications.

The invention also aims to provide compositions which use an effective amount of active compound, capable of combating microbiological contaminations of metal or mineral surfaces.

The invention aims in particular to obtain a composition comprising a compound capable both of being a good competitive inhibitor of protein attachment and of attaching effectively and in a long-lasting manner to the metal or mineral surfaces to be protected.

The invention thus proposes novel compounds capable of attaching to metal or mineral surfaces and of limiting the attachment of microorganisms, in particular bacteria, and the development of the latter on these surfaces.

The invention is in particular directed toward the compositions containing these novel compounds and their use against the microbiological contamination of metal or mineral surfaces, in the odontological field (limiting the formation of dental plaque, combating the formation of caries and periodontal diseases) and the fields of hospital hygiene and agrofoods.

A first subject of the invention is the compounds of formula (I):

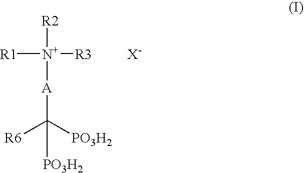

in which:
R₆ represents a hydrogen atom, a halogen atom, a linear or branched C₁-C₁₂ alkyl group, an —OH group, an amine optionally substituted with one or two linear or branched C₁-C₄ alkyl groups, or a group -A′-N⁺R′₁R′₂R′₃, X₁⁻,
R₁, R′₁, R₂, R′₂, R₃ and R′₃ represent, independently of one another:
   a linear or branched C₁-C₁₂ alkyl group, or
   an alkylammonium group —(CH₂)ₙ—N⁺RR′R″, X₂⁻ in which n is an integer between 1 and 12, and R, R′ and R″ represent, independently of one another, a linear or branched C₁ to C₄ alkyl,
A and A′ represent, independently of one another, a group —(CH₂)ₘ-Z-(CH₂)ₚ— in which:
   m is an integer between 0 and 12,
   p is an integer between 0 and 12,
   m+p is an integer between 0 and 12, and
   -Z- represents an oxygen atom, a sulfur atom, a —CR₇R₈R₉— group, a —COO— group, a —CONR₇— group or an —NR₇R₈— group in which R₇, R₈ and R₉ have the same meanings as R₆, or else an —N⁺R₄R₅—, X₃— group in which R₄ and R₅ represent, independently of one another, a linear or branched C₁-C₁₂ alkyl group,
with the condition that the molecule of formula (I) contains at least two quaternary ammonium functions,
and X, X₁, X₂ and X₃ are pharmaceutically acceptable counterions.

The term “pharmaceutically acceptable” means acceptable from a toxicity point of view.

The term “halogen atom” means a fluorine, chlorine, bromine or iodine atom.

The terms “C₁ to C₄ alkyl” and “C₁ to C₁₂ alkyl” mean, respectively, an alkyl containing 1 to 4 carbon atoms and an alkyl containing 1 to 12 carbon atoms. A linear or branched C₁ to C₄ alkyl group comprises, in particular, methyl, ethyl, n-propyl, isopropyl, n-butyl sec-butyl and tert-butyl groups.

The expression “R₇, R₈ and R₉ have the same meanings as R₆” means that R₆, R₇, R₈ and R₉ represent, independently of one another, a hydrogen atom, a halogen atom, a linear or branched C₁-C₁₂ alkyl group, an —OH group, an amine optionally substituted with one or two linear or branched C₁-C₄ alkyl groups, or a group, -A′—N⁺R′₁R′₂R′₃, X₁⁻ as defined above.

These compounds are particularly appropriate against the microbiological contamination of metal or mineral surfaces, for the following reasons:
they are nontoxic and biodegradable,
they are not necessarily biocidal,
they are not necessarily bacteriostatic,
they have a high affinity for metal surfaces (such as stainless steel or aluminum) or mineral surfaces (such as the dental surface),
they are capable of rapidly and effectively attaching in a long-lasting manner to the surfaces to be protected,
they form a self-assembled monomolecular film on contact with metal or mineral surfaces and thus create an interface between the protected surface and the outside environment, which effectively and in a long-lasting manner limits the development of biofilms on these surfaces,
this interface limits or even prevents the denaturation of the macromolecules usually responsible for the formation of biofilms on contact with the surface.

The compounds of formula (I) comprise two phosphonic groups linked to the same carbon atom (bisphosphonic or gem-diphosphonic groups) which allow attachment to the solid surfaces to be protected, and one or two optionally branched chains also linked to this same carbon atom and comprising at least two quaternary ammonium functions located one after the other on the chain or else on branched chains.

The quaternary ammonium functions have two important roles for combating the microbiological contamination phenomenon:
they make it possible to reduce the surface charge which is often very negative in the case of teeth or of metal surfaces. A weak electric charge is an important biocompatibility factor,
they allow hydration of the surface through structuring of the water at the interface with the outside medium. This hydration makes it possible to limit or even prevent the denaturation of the macromolecular compounds.

In formula (I), if R₆ represents the group -A′-N⁺R′₁R′₂R′₃, X₁⁻, then preferably A′=A, R′₁═R′₁, R′₂═R₂ and R′₃═R₃ and X₁⁻═X⁻.

R₆ preferably represents a hydrogen atom, a halogen atom, an —OH group or an amine —NH₂.

Preferably, Z represents an oxygen atom, a sulfur atom, a —CR₇R₈R₉— group, a —COO— group, a —CONR₇— group or an —NR₇R₈— group where R₇, R₈ and R₉ are as defined above. Even more preferably, Z represents an —N⁺R₄R₅—, X₃⁻ group in which R₄ and R₅ represent, independently of one another, a linear or branched C₁-C₁₂ alkyl group.

Advantageously, R₁, R₂, R₃, R₄ and R₅ represent, independently of one another, a preferably linear, C₁ to C₄ alkyl group, such as the ethyl or methyl group.

Even more advantageously, R₁, R₂ and R₃ are identical and each represent a methyl or ethyl group, and R₄ and R₅ are identical and each represent a methyl or ethyl group.

Advantageously, X, X₁, X₂ and X₃ are chosen from iodide, chloride, bromide, fluoride, sulfonate, phosphate and phosphonate ions, or any pharmacologically active ion.

Preferably, the compounds according to the present invention are chosen from those corresponding to formula (Ia) below:

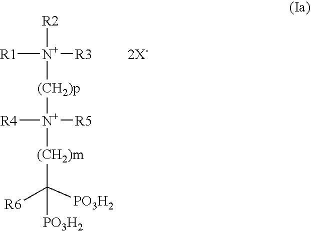

in which:
R₆ represents —OH or —NH₂,
m is an integer between 1 and 12,
p is an integer between 1 and 12,
m+p is an integer between 2 and 12,
R₁, R₂, R₃, R₄ and R₅ represent, independently of one another, a linear or branched C₁-C₄ alkyl group, and
X⁻ represents a pharmaceutically acceptable counterion.

In the compounds of formula (Ia), m is preferably between 3 and 7 and p is preferably between 1 and 4.

Advantageously, R₁, R₂, R₃, R₄ and R₅ represent, independently of one another, a preferably linear C₁ to C₄ alkyl group, such as the ethyl or methyl group.

R₁, R₂ and R₃ are preferably identical and each represent a methyl or ethyl group, and R₄ and R₅ are preferably identical and each represent a methyl or ethyl group.

Advantageously, X is chosen from iodide, chloride, bromide, fluoride, sulfonate, phosphate and phosphonate ions, or any pharmacologically active ion.

According to a second aspect, a subject of the invention is a topical oral hygiene composition comprising at least one compound of formula (I) as described above, preferably a compound of formula (Ia), preferably in combination with one or more pharmaceutically acceptable excipients.

The composition advantageously comprises between 0.001% and 10% by weight of the compound of formula (I), preferably between 0.005% and 5% by weight, even more preferably between 0.01% and 1% by weight. The composition is typically in the form of a mouthwash, a liquid spray, a toothpaste, a tooth gel, a paste to be applied, a powder, a chewing gum or gum to be applied, or a foam.

The composition can be applied to the teeth by various appropriate techniques, in particular brushing, tincture, spraying, mouthwash or chewing gum, or by means of a dental accessory such as dental floss impregnated with said composition, an optionally disposable wipe impregnated with said composition or a sponge impregnated with said composition. Other possible application means are known to those skilled in the art.

Various other ingredients can be incorporated into the composition, such as prophylactic agents, polishing agents, other surfactants, flavorings, thickeners or humectants that are suitable. It is, however, necessary to be sure that these agents do not prevent the desired attachment of the polyphosphonates to the dental surfaces.

Among the prophylactic agents, mention may be made of compounds for limiting caries, such as sodium fluoride, potassium fluoride, hexylamine hydrofluoride, but also all antiseptics and antibiotics known for their oral activity. Typically, these prophylactic agents are present in amounts sufficient, for example, to provide a fluoride ion concentration of the order of 0.5% to 2% by weight of the composition.

Among the polishing agents, mention may be made of resins (product of condensation of urea and formaldehyde), particles of resins polymerized by heating (see U.S. Pat. No. 3,070,510), silica xerogels (U.S. Pat. No. 3,538,230), precipitated silica particles, calcium pyrophosphate, insoluble sodium metaphosphate, hydrated alumina and dicalcium orthophosphate, these agents being sufficiently non-abrasive so as not to impair in an unwanted manner the surface of the tooth or of the dentine. These agents can represent, for example, 5% to 95% by weight of the composition.

Among the gelling agents or thickeners, mention may be made of natural gums, such as gum arabic, sodium carboxycellulose, or hydroxyethylcellulose, generally representing 0.5% to 10% of the composition by weight.

When the composition is in the form of an oral liquid, it typically contains an alcohol, a solubilizing agent and a nonabrasive cleansing agent, and when it is in the form of a gel, it typically comprises a thickener.

Among the humectants, mention may be made of glycerol, sorbitol, polyethylene glycol and other polyhydric alcohols, it being possible for these humectants to represent up to approximately 35% of the weight of the composition. Typically, the composition can comprise a liquid phase representing 10% to 99% by weight and comprising water and a humectant in variable proportion.

Among the flavorings, use may be made, optionally in combination, of mint oils, menthol, eugenol, orange, lemon, aniseed, vanillin or thymol, these agents generally representing less than 5% by weight of the composition.

The composition may also comprise, for example, sweetening agents (sodium saccharinate), bleaching agents (titanium dioxide or zinc oxide), vitamins, other anti-plaque agents (zinc salts, including zinc citrate, copper salts, tin salts, strontium salts, allantoin, chlorhexidine), antibacterial agents (triclosan: 2′,4,4′-10/trichloro-2-hydroxydiphenyl ether), anti-tartar agents (alkali di- and/or tetra-metal pyrophosphates), pH adjusters, dyes, anti-carie agents (caseine, urea, calcium glycerophosphates, sodium fluoride, monosodium fluorophosphate), antistaining compounds (silicone polymers), antiinflammatories (substituted salicylanilides), and desensitizing agents (potassium nitrate, potassium citrate). Other agents are mentioned in patent U.S. Pat. No. 5,258,173.

The pH of the composition is typically between 5 and 10. The pH will preferably be between 5 and 7.

### Example of Composition for a Toothpaste or a Tooth Gel (% by Weight):

bisphosphonic compound of formula (I): 0.005% to 5%
abrasive agent: 10% to 50%
thickener: 0.1% to 5%
humectant: 10% to 55%
flavoring: 0.04% to 2%
sweetening agent: 0.1% to 3%
dye: 0.01% to 0.5%
water: 2% to 45%

### Example of Composition of a Nonabrasive Gel Such as a Subgingival Gel (% by Weight):

bisphosphonic compound of formula (I): 0.005% to 5%
thickener: 0.1% to 20%
humectant: 10% to 55%
flavoring: 0.04% to 2%
sweetening agent: 0.1% to 3%
dye: 0.01% to 0.5%
water: 2% to 45%

### Example of Composition of a Mouthwash (% by Weight)

bisphosphonic compound of formula (I): 0.005% to 5%
humectant: 0% to 50%
flavoring: 0.04% to 2%
sweetening agent: 0.1% to 3%
dye: 0.01% to 0.5%
water: 45% to 95%
ethanol: 0% to 25%

A dental solution will typically comprise 90% to 99% of water. A composition of chewing gum type will typically comprise a base gum (approximately 80% to 99%), a flavoring (approximately 0.4% to 2%) and a sweetening agent (approximately 0.01% to 20%).

Those skilled in the art will incorporate, appropriately and without excessive effort, various agents as described in patent U.S. Pat. No. 6,132,702.

To prepare a dentifrice composition, the following procedure is, for example, carried out: humectants such as glycerol or propylene glycol are dispersed with the sweetening agent and water in a mixer, until the mixture becomes a homogeneous gel. A pigment, where appropriate a pH adjuster, and an anti-carie agent are then added. These ingredients are mixed until a homogeneous phase is obtained, into which phase a polishing agent is then mixed. The mixture is then transferred into a high-speed mixer, in which a thickener, a flavoring and the compound of formula (I) are mixed, under a reduced pressure of 20 to 100 mm Hg. The product obtained is a semi-solid, extrudable paste.

The dentifrice composition is typically applied regularly, each day or every two or three days, from one to three times a day, at a pH of approximately 5 to 9 or 10, in general between 5.5 and 8.

A subject of the present invention is also a cosmetic process for preventing the appearance of dental plaque or limiting the development of dental plaque on the teeth, comprising the application of an effective amount of the oral hygiene composition as described above, to the teeth. An “effective amount” means an amount that makes it possible to limit or prevent the appearance or the development of dental plaque.

A subject of the present invention is also a medicament comprising at least one compound of formula (I), preferably (Ia), or else the oral composition described above, in particular for preventing the formation of caries or for preventing periodontal diseases.

According to a third aspect, a subject of the present invention is an anticontamination composition intended to prevent or limit the attachment of macromolecules to solid surfaces, such as metal or mineral surfaces, comprising at least one compound of formula (I) as described above, preferably at least one compound of formula (Ia).

In the context of the present invention, the term “macromolecule” is intended to mean an organic molecule that has a relatively high molecular mass (molecular weight greater than 1000 Da) and that can serve as a substrate for the attachment and development of micro-organisms on solid surfaces. These macromolecules are in particular of peptide, protein, polysaccharide, polyphenolic, lipid or nucleic acid type.

In addition, the anticontamination composition according to the invention limits or prevents the attachment of microorganisms, in particular of bacteria, and thus limits or prevents the formation and the development of a biofilm on solid surfaces, in particular metal or mineral surfaces.

In the context of the present invention, the term “microorganism” denotes in particular bacteria, viruses and prions.

Among the bacteria targeted by this type of composition, mention may in particular be made of Streptococcus (mutans, sanguis, pyogenes, etc.), Salmonella, Listeria monocytogenes, Legionella, Vibrio cholerae, Lactobacillus, Porphyromonas, Staphylococcus (aureus, epidermidis), Pseudomonas, Escherichia coli and Candida.

The anticontamination composition advantageously comprises between 0.001% and 10% by weight of the compound of formula (I), preferably the compound of formula (Ia), preferably between 0.005% and 5% by weight, even more preferably between 0.01% and 1% by weight.

The pH of the composition is typically between 5 and 10. The pH will preferably be between 5 and 7. This product may be used in a mixture as an additive in detergent or disinfectant formulations used industrially.

The chemical agents most commonly used for the manufacture of detergents are surfactants (ionic, nonionic or amphoteric), chelating agents, alkalis and solvents. These formulations can also contain active ingredients of antiseptic, biocide or antibiotic type.

By way of example, the compound of formula (I) can be incorporated into a formulation having the following composition:
disinfectants (such as glutaraldehyde, peracetic acid, sodium hypochlorite, etc.) representing, for example, 0.01% to 30% by weight of the composition,
surfactants (such as etholated, propoxylated fatty alcohols, amine oxides, condensates of ethylene oxide and of propylene oxide, quaternary ammonium salts, sulfates, sulfonates and sulfosuccinates) representing, for example, 0.01% to 30% by weight of the composition,
chelating agents (for example, EDTA, sodium imino-disuccinate, sodium carbonates, orthophosphates and silicates, condensed phosphates) representing, for example, 0.1% to 5% by weight of the composition,
alkalis (carbonates, phosphates and silicates) representing, for example, from 0.1% to 40% by weight of the composition,
water-miscible solvents (alcohols, glycol) or water-immiscible solvents (turpentine derivatives, petroleum derivatives), which are optionally ionic, representing, for example, 0.1% to 80% by weight of the composition.

The surfaces that can be protected by the anticontamination composition according to the invention are, for example, metal surfaces such as iron, stainless steel, chromium, aluminum, zinc, titanium, tungsten, lead or copper, and also alloys or composites containing at least one of these metals, or else mineral surfaces, such as silicon and its derivatives, silicious materials, or calcic, ceramic or dental surfaces.

The application of the anticontamination composition to the surfaces to be treated can be carried out by soaking or immersion of this surface in the composition, or by spraying the composition onto the surface to be treated. It can also be carried out by means of accessory products, for instance the use of optionally disposable wipes impregnated with the composition.

The subject of the present invention is thus the use of the compounds of formula (I) as described above or of the anti-microbiological contamination compositions as described above, for limiting or preventing the attachment of macromolecules, of microorganisms and of a biofilm to solid surfaces, in particular metal or mineral surfaces.

These surfaces are, for example, the surface of industrial, agrofoods or hospital equipment, of land, air or sea buildings, constructions or vehicles, or of air-conditioning or refrigeration equipment, or else the surface of surgical instruments, of prostheses, of dentistry instruments or of biological and medical sensors.

Example of a Liquid Anti-Microbiological Contamination Composition Applied by Soaking, Rinsing, Depositing with a Wet Cloth or by Sprinkling (% by Weight):
compound of formula (I): 0.02% to 5%
water: 15% to 99%
ethanol: 0% to 85%.

In the above composition, the presence of alcohol facilitates the wetting of the surface and the homogeneity of the coating. Furthermore, the evaporation of the alcohol makes it possible to obtain very high concentrations at the surface, thereby facilitating rapid adsorption. The ethanol can advantageously be replaced with water-miscible (C₁ to C₆ alcohols, in particular isopropyl alcohol, aldehydes, ketones, including acetone, ethers, etc.) or water-immiscible (C₄ to C₈ alkanes in particular) volatile compounds.

Such a composition leaves only a few solid residues after evaporation of the liquids and is therefore particularly indicated when the surface must be directly used after application, in particular for apparatus and instruments that have to come into contact with food or the human organism.

### Example of a Liquid, Surface-Rinsing Composition (% by Weight):

bisphosphonic compound of formula (I): 0.02% to 5%
humectant: 0% to 50%
odorizing agent: 0.04% to 2%
dye: 0% to 0.5%
water: 15% to 99%
ethanol: 0% to 85%.

In this composition intended to facilitate the maintenance of esthetic surfaces, such as stainless steel sheets in public buildings, the presence of humectants, odorizing agents or even dyes is envisioned in order to render the application easier, but also to improve the esthetic attractiveness (for example, fluorescent brightening dye).

### Example of Composition for a Paste or a Gel (% by Weight):

bisphosphonic compound of formula (I): 0.02% to 5%
abrasive agent: 10% to 50%
thickener: 0.1% to 5%
humectant: 10% to 55%
dye: 0% to 0.5%
pH modifier: 0% to 3%
water: 2% to 60%.

This composition is especially intended for scouring highly fouled surfaces, in particular on vertical, or even inverted, surfaces.

### Example of a Foam-Type Composition (% by Weight)

bisphosphonic compound of formula (I): 0.02% to 5%
surfactant: 0% to 20%
thickener: 0% to 20%
water: 25% to 50%
ethanol: 0% to 25%
propellant: 5% to 70%.

In this composition, the propellant may be a liquefied gas such as alkanes (propane or butane), fluorocarbon-based products (F14, F26, etc.), pressurized gases (CO₂, N₂, etc.) or even volatile liquids. This formulation is particularly advantageous when the parts are difficult to reach (inside of narrow tubing, heat exchangers, air-conditioners, etc.). It is also advantageous for uses with large surfaces (fermenters in biotechnology, rooms for preparation or cutting up in agrofoods, etc.).

### Example of a Powder-Type Composition (% by Weight)

bisphosphonic compound of formula (I): 0.02% to 5%
surfactant: 5% to 95%
complexing agent: 1% to 10%
diluent: 10% to 90%
humectant: 1% to 5%
dye: 0% to 0.5%
pH modifier: 0% to 3%.

In this composition, the overall composition may be that of a washing powder already known to those skilled in the art, to which the bisphosphonic compound of formula (I) is added. This formulation is particularly advantageous as a washing agent for dishwashers and washing machines.

In all the compositions above, the bisphosphonic compounds of formula (I) can be used either alone or in combination.

### Example of a Composition for Coating Prostheses (% by Weight):

bisphosphonic compound of formula (I): 0.02% to 5%
complexing agent: 0% to 10%
pH modifier: 0% to 3%.

In this composition, the addition of complexing agents and pH modifiers is intended to ensure optimal attachment of the bisphosphonic compound of formula (I). Furthermore, the use of a bisphosphonic compound of formula (I) bound to one or more biologically active molecules, of peptide, protein, lipid, carbohydrate or nucleic acid type, can make it possible to obtain better biocompatibility or to orient the organisms reaction. The prostheses concerned are in particular metal systems (stainless steel, nitinol, titanium, nickel-chromium, etc.) either to be in contact with tissues while at the same time remaining outside the organism (such as dental or auditory prostheses) or to be implantable, at the vascular level, bone level, dental implants, etc.

### Example of a Composition for Coating Sensors (% by Weight):

bisphosphonic compound of formula (I): 0.02% to 5%
complexing agent: 0% to 10%
pH modifier: 0% to 3%.

In this application also, the use of a bisphosphonic compound of formula (I) bound to one or more biologically active molecules, of peptide, protein, lipid, carbohydrate, nucleic acid or other type, makes it possible to detect a molecular, particulate, cellular, viral, etc. object, the concentration of which can be assayed.

According to the uses selected, the anti-microbiological contamination compositions according to the invention are typically applied after each use (for instance with surgical equipment), after each cycle of use (for instance with equipment for industrial cutting up of meat), regularly during general maintenance (esthetic surfaces), or even just once.

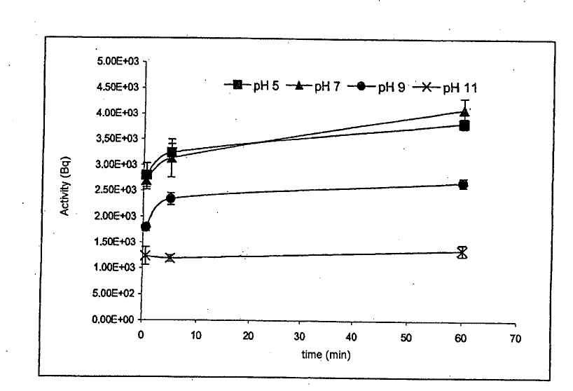

FIG. 1 represents the activity (in Becquerel) of the model bisphosphonic molecules attached to the support as a function of the contact time.

| | |
|---|---|
| ▪ | pH = 5 |
| ♦ | pH = 7 |
| ▾ | pH = 9 |
| x | pH = 11 |

The present invention is illustrated by the following examples.

### A) Synthesis of Compounds According to Formula (I)

### A-1) Step 1

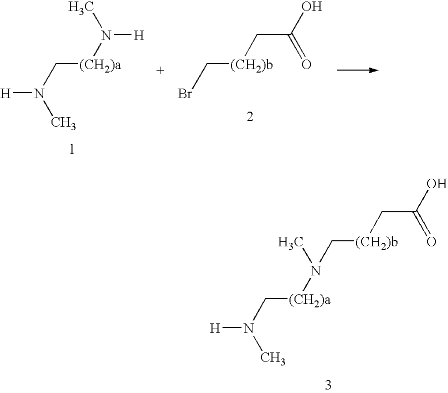

Five equivalents of the diamine 1 are dissolved in a minimum of acetonitrile. The bromoacid 2 is then added dropwise. The mixture is stirred for 3 to 24 h. The excess diamine is separated by recrystallization or by washing under hot conditions. Drying is then carried out.

| Molecule 3 | Step 1 |
|---|---|
| b | 3 |
| a | 1 |
| yield | 98% |
| m₁ (g) | 9.0 g |
| m₂ (g) | 4.0 g |
| m₃ (g) | 3.2 g |

In step 1, a tetramethylated diamine of formula (CH₃)₂N—CH₂—(CH₂)ₐ—N(CH₃)₂ can also be used in place of the dimethylated diamine 1. In this case, fewer equivalents of alkyl halide will be used in example 3.

### A-2) Step 2

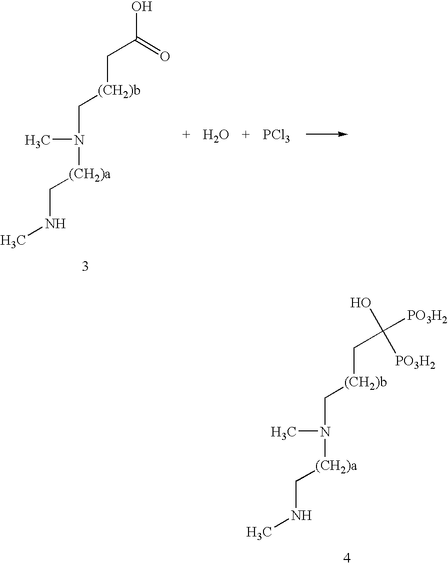

The starting product 3 is dispersed in chlorobenzene.

2.5 equivalents of H₂O are added to this mixture. The mixture is heated to 40° C.

Phosphorus trichloride is then added dropwise by means of a dropping funnel.

The mixture is refluxed for two hours.

The reaction is stopped by adding an excess of water.

The product 4 thus formed is refluxed (100° C.) for two hours.

The molecule 4 is purified by crystallization from ethanol.

| Molecule 4 | Step 2 |
|---|---|
| b | 3 |
| a | 1 |
| yield | 50% |
| m₃ | 1.00 g |
| mPCl₃ | 1.26 g |
| m₄ | 0.85 g |

### A-3) Step 3

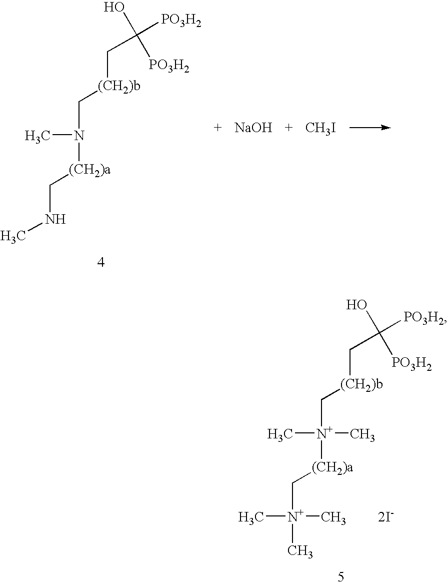

The molecule 4 is taken up in an excess of CH₃I, to which three equivalents of anhydrous sodium hydroxide are added. The mixture is stirred in the dark and without being exposed to air, at reflux for 24 to 72 hours.

The product formed is condensed under vacuum.

| Molecule 5 | Step 3 |
|---|---|
| b | 3 |
| a | 1 |
| yield | 30% |
| m₄ | 0.27 g |
| mCH₃I | 0.09 g |
| m₅ | 0.10 g |

Step 3 can also be carried out by reacting another alkyl halide in place of the methyl iodide, for instance methyl bromide.

### A-4) Other Syntheses

Moreover, molecules in which the number of quaternary ammonium functions is greater than two can readily be obtained by adding stoichiometric amounts of bromoalkyltrialkylammonium bromide to an aminoalkyl diphosphonate. This step may or may not be followed by methylation with iodomethane according to the degree of saturation of the substituted amine.

For example, molecules comprising three or four ammonium functions can be synthesized in the following way:

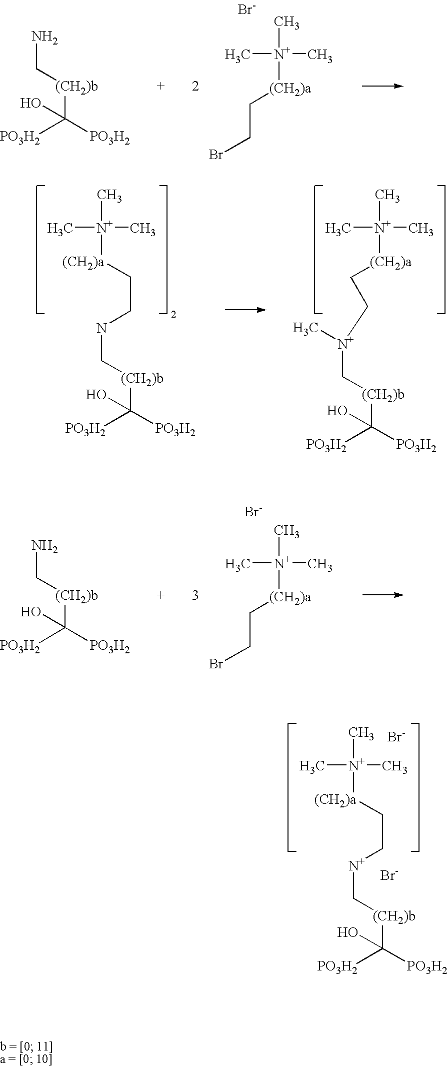

In general, those skilled in the art will be able to prepare the compounds of formula (I) without difficulty, by using conventional and well-known syntheses thereof.

### B) Attachment of Bisphosphonic Molecules to a Hydroxyapatite Support

Tests were carried out on radiolabeled model molecules in order to demonstrate the ability of the bisphosphonic molecules to attach rapidly and homogeneously to mineral surfaces. These tests, carried out under different pH conditions, made it possible to demonstrate rapid kinetics for attachment of the bisphosphonic acids to mineral surfaces (hydroxyapatite).

### B-1) Materials and Methods

### B-1-1) Solutions

Aqueous solutions of bisphosphonic acids radiolabeled with iodine 125 were prepared at concentrations of 0.1 mol·l⁻¹ and 0.01 mol·l⁻¹. The pH of these solutions was adjusted to 5, 7, 9 and 11 using molar solutions of HCl and NaOH. Each solution received an amount of radiolabeled molecules corresponding to 5×10⁸ Bq/ml.

### B-1-2) Surface

The surfaces serving as a support for the bisphosphonic compounds consist of hydroxyapatite powder (CHT® ceramic hydroxyapatite, calcium phosphate (Ca₅(PO₄)₃OH)₂, Biorad, France). This surface is packaged in hemolysis tubes at a rate of 14 mg per tube.

### B-1-3) Coating of Surfaces

The molecules serving to coat the surfaces are synthetic bisphosphonic compounds, the structure of which is the following:

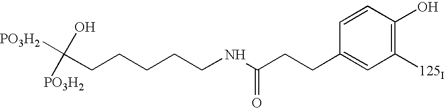

These molecules were selected as a model for the attachment of bisphosphonic acids to metal and mineral surfaces because of their structural similarity (presence of a bisphosphonic group and of a nitrogen atom), but also because of their physicochemical properties (high water-solubility, rapid adsorption onto the surfaces under consideration) similar to those of the molecules corresponding to general formula (I). A volume of 200 μl of coating solution is added to each tube containing the surfaces. A control is carried out using 200 μl of sterile distilled water (pH 6.8±0.2). The incubation times used for the attachment of the molecules of bisphosphonic compounds are 30 seconds, 5 minutes or 1 hour. The supernatant is then removed, taking care not to draw up the particles of surface, and then two cycles of rinsing/decanting/removing the supernatant are carried out using 3 ml of distilled water.

### B-1-4) Counting the Attached Molecules

After the rinsing water has been removed, the gamma-radioactivity emitted during the disintegration of the ¹²⁵I present on the bisphosphonic compounds attached to the surface of the hydroxyapatite beads is counted using a Cobra 2 autogamma counting system (Packard Bioscience Company, France).

### B-2) Results

The results are expressed as a function of the pH. The activity in Becquerel is measured as a function of the contacting time, for a bisphosphonic compound concentration of 0.1 mol/l. The results are given in FIG. 1.

A strong influence of the pH, both on the attachment kinetics and on the amount of product attached per unit of surface, is noted. It is noted that the attachment kinetics rapidly reach a plateau phase since, from the first five minutes of contacting onward, it is noted that the available surface is almost entirely coated. Moreover, the pH has a considerable influence on the charge of the molecule and brings about a strong electrostatic repulsion at high pHs, which explains the lower coating rates observed at pH 9 and 11.

The preferred pH at which the bisphosphonic compounds are used for coating a surface is therefore between 5 and 7.

### C) Prevention of Surface Contamination

### C-1) Materials and Methods

C-1-1) Surfaces

The surfaces serving as a support for the biofilm consist of hydroxyapatite powder (CHT® ceramic hydroxyapatite, calcium phosphate (Ca₅(PO₄)₃OH)₂, Biorad, France). The particle size of the hydroxyapatite powder (HAP) is 80±8 μm and the developed surface area is 72 cm²·g⁻¹.

This surface is packaged in hemolysis tubes at a rate of 14 mg per tube. The corresponding amount of particles of surface per tube is 5000. Before use, they are sterilized by dry heat (oven, Tau, Italy) at 180° C. for 2 hours.

C-1-2) Coating Molecules

The molecules serving to coat the surfaces are synthetic bisphosphonic acids. The molecule used is molecule A, synthesized in example A and having the following formula:

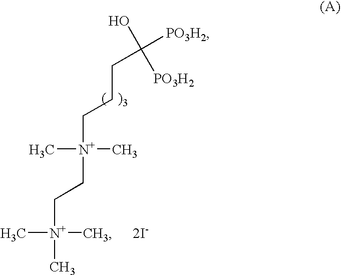

Solutions are prepared at 0.1 mol·l⁻ (pH 4.8) and sterilized by filtration through a 0.2 μm filter (Minisart, Sartorius, France).

C-1-3) Artificial Saliva

In order to reproduce the oral environment, the following saliva model was formulated (according to Hutteau & Mathlouti, 1998):

Artificial saliva: NaHCO₃ 5.208 g·l⁻¹; KH₂PO₄.3H₂O 1.369 g·l⁻¹; NaCl 0.877 g,l⁻¹; NaN₃ 0.500 g·l⁻¹; KCl 0.477 g·l⁻¹; CaCl₂.2H₂O 0.441 g·l⁻¹; mucin at 2.16 g·l⁻¹ and alpha amylase at 200 000 IU·l⁻¹.

The artificial saliva is adjusted to isotonic pH (pH 7) and sterilized by filtration through a 0.2 μm filter (Minisart, Sartorius, France).

C-1-4) Bacterial Strain

The study is carried out on a model of cariogenic bacteria: Streptococcus mutans ATCC 25175D (LGC Promochem, Molscheim, France). Streptococcus mutans is a constituent of dental plaque and a major etiological agent in dental caries.

The strain is stored in aliquots at −80° C. It is placed in culture again by transferring a 2 ml pipette tip to a 10 ml tube of Schaedler broth (Bio Mérieux, France) and incubating at 37° C. for 24 hours. The absorbance of a 1/20 dilution of the stock solution obtained is measured at 600 nm and it is then diluted in artificial saliva (prepared as indicated in C-1-3) so as to obtain 3 ml of suspension adjusted (SA) to approximately 5×10⁶ colony-forming units (cfu) in 100 μl.

C-1-5) Coating of Surfaces

A volume of 200 μl of coating solution (C-1-2) is added to each tube containing the surfaces (C-1-1). A control is carried out using 200 μl of sterile distilled water (pH 6.8±0.2). The incubation time used for the attachment of the bisphosphonic molecules is 3 minutes at 37° C. The supernatant is then removed, taking care not to draw up the particles of surface, and then two cycles of rinsing/decanting/removing the supernatant are carried out using 3 ml of sterile distilled water.

C-1-6) Formation of the Biofilm

A volume of 3 ml of artificial saliva (C-1-3) is added to the tubes containing the coated surfaces (controls and tests). Immediately following this, a volume of 100 μl of the adjusted bacterial suspension (C-1-4) is inoculated into the tubes. The tubes are incubated for 4 to 24 hours at 37° C.

C-1-7) Counting of the Bacteria of the Biofilm

The colonized particles are washed in order to remove the nonadherent bacteria by performing three cycles of rinsing/decanting/removing the supernatant (taking care not to draw up the particles of surface) using 3 ml of physiological saline. The particles of surface are resuspended in 1 ml of physiological saline and treated with ultrasound in order to detach the adherent bacteria (Branson 1200, 47 KHz, 95 W, 5 minutes, Bransonic, USA). The bacterial suspension obtained is counted by means of ten-fold dilutions in physiological saline and plating out of 100 μl of the dilutions −1 and −2 on blood agar. The counting is carried out after 48 to 72 hours of incubation at 37° C. The number of bacteria is expressed in cfu per 14 mg of hydroxyapatite.

### C-2) Results

C-2-1) Prevention of Bacterial Contamination

Coating of the HAP surfaces is carried out for 3 minutes with the solution to be tested. The HAP surfaces are then incubated for 4 to 24 hours with a bacterial solution.

Table 1 gives the results obtained, showing the bacterial colonization as a function of time for uncoated hydroxyapatite surfaces or hydroxyapatite surfaces coated with the bisphosphonic compounds (control and test, respectively). For the statistical analysis, the numbers of cfu were converted to log₁₀ in order to obtain a normal distribution of the results. The Student's test was used to evaluate the significance of the values in the two experiments (table I).

TABLE I
Mean values of the counts of Streptococcus mutans
adhering to the hydroxyapatite

| Time (hours) | 4 | 8 | 15 | 24 |
|---|---|---|---|---|
| Control (C) | 5.19 ± 0.25 | 5.73 ± 0.18 | 7.18 ± 0.40 | 7.04 ± 0.25 |
| Test (T) | 4.72 ± 0.19 | 4.98 ± 0.32 | 5.02 ± 0.24 | 5.07 ± 0.30 |
| Log decrease | 0.47 | 0.75 | 2.16 | 1.97 |
| Probability P | 0.060 | 0.024 | 0.001 | 0.001 |

Test: hydroxyapatite (HAP) coated with the bisphosphonic compounds. Control: Uncoated HAP. Data expressed in log of the number of cfu per 14 mg of HAP (n=6).

Growth of the biofilm is observed in the two cases, but the colonization curve profile is not similar. The number of bacteria colonizing the hydroxyapatite surfaces is higher for the control. The difference in colonization is significant from 6 hours and after 8, 15 and 24 hours of incubation (P<0.05). Thus, after 15 hours, the number of bacteria coating the HAP surfaces coated with the bisphosphonic compounds decreases by a factor of 100.

### D) Evaluation of the Bactericidal Activity of Molecule A

### D-1) Principle

The bactericidal activity of molecule A is evaluated according to the protocol of Standard NF EN 1040 modified so as to take into account the practical operating conditions. The conditions selected are the following: target strain, Streptococcus mutans; medium, physiological saline; temperature, 37° C.; contact time, 5 minutes.

### D-2) Materials and Methods

D-2-1) Solutions of Molecule A

A stock solution at 2×10⁻¹ mol·l⁻¹ (pH adjusted to 6.0±0.1) is prepared and sterilized by filtration through a 0.2 μm filter (Minisart, Sartorius, France). A standard range of solutions is then prepared by making dilutions in sterile distilled water, i.e. 10⁻¹, 2×10⁻² and 10⁻² mol·l⁻¹.

D-2-2) Bacterial Strain

The tests are carried out with the cariogenic bacteria model chosen for the biofilm study: Streptococcus mutans ATCC 25175D (LGC Promochem, Molscheim, France). The strain is stored in aliquots at −80° C. It is placed in culture again by transferring a 2 ml pipette tip to a 13 ml tube of Schaedler broth (Bio Mérieux, France) and incubating at 37° C. for 24 hours.

The absorbance of a 1/20th dilution of the stock solution obtained is measured at 600 nm and it is then diluted in physiological saline or in artificial saliva so as to obtain 3 ml of suspension adjusted (SA) to approximately 10⁷ colony-forming units (cfu) in 100 μl.

D-2-3) Measurement of the Bactericidal Activity

For each of the concentrations of molecules tested, 2 tubes are filled with 900 μl of physiological saline. 100 μl of SA prepared in the medium are then added immediately before the tests. Finally, at time T₀, 1 ml of the concentration of molecules or sterile distilled water (control) is added to the tubes. The latter are incubated at 37° C. for 5 minutes±15 seconds. The concentration range of molecules in contact with the bacteria is therefore the following: 10⁻¹, 5×10⁻², 10⁻² and 5×10⁻³ mol·l⁻¹. After incubation, the number of bacteria present in the tubes is determined by means of ten-fold dilutions in physiological saline (Bio Mérieux, France) and plating out of 100 μl of the dilutions −2, −3 and −4 on blood agar (Columbia+5% of sheep blood, Bio Mérieux, France). The counting is carried out after 48 hours of incubation at 37° C. The number of bacteria is expressed in cfu per ml.

### D-3) Results

Table II gives the results obtained for the physiological saline medium.

TABLE II
Results of the tests to measure the bactericidal activity of molecule
A on S. mutans ATCC 25175D in physiological saline.

| | Concentration | | |
|---|---|---|---|
| Concentration | in mg · l⁻¹ | | Log₁₀ cfu |
| in mol · l⁻¹ | (ppm) | cfu per ml | per ml |
| 0.1 | 51 900 | 2.76 × 10⁶ | 6.44 |
| 0.05 | 25 950 | 1.93 × 10⁶ | 6.29 |
| 0.01 | 5190 | 2.56 × 10⁶ | 6.41 |
| 0.005 | 2595 | 2.57 × 10⁶ | 6.41 |
| 0 (control) | 0 | 2.91 × 10⁶ | 6.46 |

No significant decrease in the bacterial population is observed in the tubes containing the molecule compared with the control tube. The molecule does not therefore show any bactericidal activity at the maximum concentration tested, i.e. 10⁻¹ mol·l⁻¹.

What is claimed is:

1. A bisphosphonic compound of formula (I): 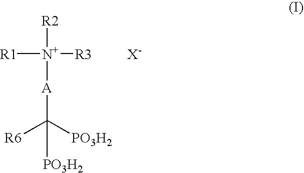
in which:
R₆ represents a hydrogen atom, a halogen atom, a linear or branched C₁-C₁₂ alkyl group, an —OH group, an amine optionally substituted with one or two linear or branched C₁-C₄ alkyl groups, or a group -A′—N⁺R′₁R′₂R′₃, X₁⁻,
R₁, R′₁, R₂, R′₂, R₃ and R′₃ represent, independently of one another:
a linear or branched C₁-C₁₂ alkyl group, or
an alkylammonium group —(CH₂)ₙ—N⁺RR′R″, X₂⁻ in which n is an integer between 1 and 12, and R, R′ and R″ represent, independently of one another, a linear or branched C₁ to C₄ alkyl,
A and A′ represent, independently of one another, a group —(CH₂)ₘ-Z-(CH₂)ₚ— in which:
m is an integer between 0 and 12,
p is an integer between 0 and 12,
m+p is an integer between 0 and 12, and
-Z- represents an oxygen atom, a sulfur atom, a —CR₇R₈R₉— group, a —COO— group, a —CONR₇— group or an —NR₇R₈— group in which R₇, R₈ and R₉ have the same meanings as R₆, or else an —N⁺R₄R₅—, X₃⁻ group in which R₄ and R₅ represent, independently of one another, a linear or branched C₁-C₁₂ alkyl group,
with the condition that the molecule of formula (I) contains at least two quaternary ammonium functions,
and X, X₁, X₂ and X₃ are pharmaceutically acceptable counterions.

2. A bisphosphonic compound as claimed in claim 1, characterized in that R₆ represents a hydrogen atom, a halogen atom, an —OH group or an amine —NH₂.

3. A bisphosphonic compound as claimed in claim 1 or 2, characterized in that Z represents an oxygen atom, a sulfur atom, a —CR₇R₈R₉— group, a —COO— group, a —CONR₇— group or an —NR₇R₈— group where R₇, R₈ and R₉ are as defined in claim 1.

4. A bisphosphonic compound as claimed in any one of claims 1 to 3, characterized in that Z represents an —N⁺R₄R₅—, X₃⁻ group in which R₄ and R₅ represent, independently of one another, a linear or branched C₁-C₁₂ alkyl group.

5. A bisphosphonic compound as claimed in any one of claims 1 to 4, characterized in that R₁, R₂, R₃, R₄ and R₅ represent, independently of one another, a linear or branched C₁-C₄ alkyl group, preferably a methyl or ethyl group.

6. A bisphosphonic compound as claimed in claim 1, characterized in that it corresponds to formula (Ia) below: 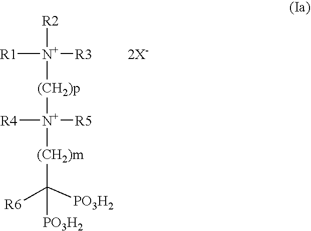
in which:
R₆ represents —OH or —NH₂,
m is an integer between 1 and 12, preferably between 3 and 7,
p is an integer between 1 and 12, preferably between 1 and 4,
m+p is an integer between 2 and 12,
R₁, R₂, R₃, R₄ and R₅ represent, independently of one another, a linear or branched C₁-C₄ alkyl group, preferably a methyl or ethyl group, and
X⁻ represents a pharmaceutically acceptable counterion such as iodide, chloride, bromide, fluoride, sulfonate, phosphate or phosphonate ions, or any pharmacologically active ion.

7. A topical oral hygiene composition, characterized in that it comprises at least one compound of formula (I) as claimed in any one of claims 1 to 6 in combination with one or more pharmaceutically acceptable excipients.

8. The composition as claimed in claim 7, characterized in that the compound of formula (I) has a concentration of between 0.001% and 10% by weight, more preferably between 0.005% and 5% by weight, and even more preferably between 0.01% and 1% by weight.

9. The composition as claimed in claim 7 or 8, characterized in that it is in the form of a mouthwash, a liquid spray, a toothpaste, a tooth gel, a paste to be applied, a liquid to be applied, a powder, a chewing gum or gum to be applied, or a foam.

10. A dental accessory, such as a dental floss, an optionally disposable wipe or a sponge, characterized in that it is impregnated with the composition as claimed in any one of claims 7 to 9.

11. An anti-microbiological contamination composition for metal or mineral surfaces, characterized in that it comprises a compound of formula (I) as claimed in any one of claims 1 to 6.

12. The composition as claimed in claim 11, characterized in that it comprises between 0.001% and 10% by weight, preferably between 0.005% and 5% by weight, of the compound of formula (I).

13. An optionally disposable wipe impregnated with the composition as claimed in claim 11 or 12.

14. The use of the compounds of formula (I) as claimed in any one of claims 1 to 6, for preventing or limiting the attachment of macromolecules to solid surfaces such as metal or mineral surfaces.

15. The use of the compounds of formula (I) as claimed in any one of claims 1 to 6, for preventing or limiting the attachment of microorganisms, preferably of bacteria, to solid surfaces such as metal or mineral surfaces.

16. The use of the compounds of formula (I) as claimed in any one of claims 1 to 6, for preventing or limiting the formation and the development of a biofilm on solid surfaces, in particular metal or mineral surfaces.

17. The use of the compounds of formula (I) as claimed in any one of claims 14 to 16, characterized in that the metal surfaces belong to the group comprising iron, stainless steel, chromium, aluminum, zinc, titanium, tungsten, lead or copper, and also alloys or composites containing at least one of these metals, and the mineral surfaces belong to the group comprising silicon and its derivatives, silicious materials, calcic surfaces, ceramic surfaces or dental surfaces.

18. The use as claimed in any one of claims 14 to 17, characterized in that the surfaces are the surface of industrial, agrofoods or hospital equipment, of land, air or sea buildings, constructions or vehicles, or of air-conditioning or refrigeration equipment, or else the surface of surgical instruments, of prostheses, of dentistry instruments or of biological and medical sensors.

19. A process for preventing the appearance of dental plaque or limiting the development of dental plaque on the teeth, comprising the application of an effective amount of a composition as claimed in any one of claims 7 to 9, to the teeth.

20. A medicament comprising at least one compound as claimed in any one of claims 1 to 6.

21. The medicament as claimed in claim 20, for the prevention of caries or periodontal diseases.
